(19) United States
(12) Patent Application Publication
(10) Pub. No.: US 2026/0020771 A1
(43) Pub. Date: Jan. 22, 2026

(54) METHOD FOR QUANTIFYING BLOOD FLOW VOLUME BASED ON A BLOOD FLOW MODEL AND SYSTEM SUPPORTING THE SAME

(71) Applicants: KNU-INDUSTRY COOPERATION FOUNDATION, Chuncheon-si (KR); THE ASAN FOUNDATION, Seoul (KR); UNIVERSITY OF ULSAN FOUNDATION FOR INDUSTRY COOPERATION, Ulsan (KR)

(72) Inventors: Ho Jin Ha, Chuncheon-si (KR); Gyu Han Lee, Seoul (KR); Dong Hyun Yang, Seoul (KR)

(73) Assignees: KNU-INDUSTRY COOPERATION FOUNDATION, Chuncheon-si (KR); THE ASAN FOUNDATION, Seoul (KR); UNIVERSITY OF ULSAN FOUNDATION FOR INDUSTRY COOPERATION, Ulsan (KR)

(21) Appl. No.: 19/273,991

(22) Filed: Jul. 18, 2025

(30) Foreign Application Priority Data
Jul. 19, 2024 (KR) 10-2024-0095990

(51) Int. Cl. A61B 5/026; G06T 7/00; G06T 7/10; G16H 30/40; G16H 50/20; G16H 50/50

(52) U.S. Cl. CPC A61B 5/026; G06T 7/0014; G06T 7/10; G16H 30/40; G16H 50/20; G16H 50/50; G06T 2207/10076; G06T 2207/10088; G06T 2207/30104

(57) ABSTRACT

A blood flow volume quantification system based on a blood flow modeling includes: an image receiver configured to receive 4D flow magnetic resonance imaging (MRI) images and MRI images of a plurality of non-diseased individuals; a representative model generation unit configured to perform vessel segmentation based on the received MRI images and to designate a representative model from among the MRI images in which vessel segmentation has been performed, based on a predetermined criterion; an integrated model generation unit configured to register MRI images of other non-diseased individuals to the representative model to generate an integrated model; and a standard blood flow model generation unit configured to register the 4D flow MRI images to the integrated model to generate a standard blood flow model.

### CROSS-REFERENCE TO RELATED APPLICATION

This application claims the benefit under 35 U.S.C. § 119 (a) of Korean Patent Application No. 10-2024-0095990, filed on Jul. 19, 2024, the entire disclosure of which is incorporated herein by reference for all purposes.

### BACKGROUND

### 1. Field

The present disclosure relates to a method for quantifying blood flow volume based on a blood flow model and a system supporting the same.

### 2. Description of Related Art

Research is actively underway on disease diagnosis technologies that utilize four-dimensional flow Magnetic Resonance Imaging (hereinafter referred to as “4D flow MRI”), which measures time-varying blood flow in three-dimensional space based on the inherent sensitivity of MRI to blood flow.

Since 4D flow MRI enables three-dimensional velocity measurement with temporal resolution, it allows for real-time measurement of the direction, velocity, pressure gradient, and other related hemodynamic characteristics of blood flow in a patient's heart and major blood vessels. Accordingly, it enables precise visualization and analysis of blood flow patterns in the major vessels, thereby allowing medical professionals to utilize the results in disease diagnosis and treatment planning.

However, in actual clinical environments, analyzing and interpreting the vast amount of hemodynamic data requires considerable expertise and time, and maintaining accuracy during data processing is also challenging, which hinders the effective application of 4D flow MRI.

Furthermore, conventional 4D flow MRI systems are limited in effectively distinguishing subtle differences in blood flow patterns that may occur under various disease conditions, which leads to reduced diagnostic accuracy, particularly in the early stages of disease. This reduction in accuracy can result in delayed or incorrect diagnoses, leading to suboptimal treatment planning, postponed therapeutic intervention, and potentially poorer patient outcomes.

### SUMMARY

The present disclosure is directed to solving the aforementioned problems. In particular, an object of the present disclosure is to provide a method for quantifying blood flow volume based on a blood flow model, in which a subject's blood flow is quantified using a standard blood flow model for 4D flow MRI that is generated in advance based on data collected from healthy individuals. This approach addresses the complexity of data post-processing and interpretation associated with conventional 4D flow MRI and enables the diagnosis and treatment of vascular diseases in the subject.

The object of the present disclosure is not limited to the above, and other objects not explicitly mentioned will be clearly understood by those skilled in the art from the following description.

In one general aspect, a blood flow volume quantification system based on a blood flow model according to an example of the present disclosure includes an image receiver configured to receive 4D flow magnetic resonance imaging (MRI) images and MRI images of a plurality of non-diseased individuals; a representative model generation unit configured to perform vessel segmentation based on the received MRI images and to designate a representative model from among the MRI images in which vessel segmentation has been performed, based on a predetermined criterion; an integrated model generation unit configured to register MRI images of other non-diseased individuals to the representative model to generate an integrated model; and a standard blood flow model generation unit configured to register the 4D flow MRI images to the integrated model to generate a standard blood flow model. The 4D flow MRI images and the MRI images are images of a body region captured for diagnostic purposes.

The representative model generation unit may be configured to analyze the MRI images to separate fat and water in the aorta, and segments the vessels by defining vessel boundaries based on the separation.

The blood flow volume quantification system based on a blood flow model may further include a blood flow image display unit configured to compare a velocity vector of the generated standard blood flow model with a blood flow velocity vector of a 4D flow MRI image of a subject, and to display, based on a result of the comparison, an abnormal blood flow region of the subject on the 4D flow MRI image of the subject.

The blood flow image display unit may be configured to compare an angle between the velocity vector of the standard blood flow model and the blood flow velocity vector of the 4D flow MRI image of the subject, and to determine whether the angle exceeds a predetermined angular threshold.

The blood flow image display unit may be configured to visually display an analysis result obtained by interpolating the 4D flow MRI image of the subject into a plurality of time steps at predetermined intervals.

In another general aspect, a method for quantifying blood flow volume based on a blood flow model according to an example of the present disclosure includes receiving 4D flow magnetic resonance imaging (MRI) images and MRI images of a plurality of non-diseased individuals; performing vessel segmentation based on the received MRI images; designating a representative model based on a predetermined criterion from among the MRI images in which vessel segmentation has been performed; registering MRI images of other non-diseased individuals to the representative model to generate an integrated model; and registering the 4D flow MRI images to the generated integrated model to generate a standard blood flow model.

The step of performing vessel segmentation based on the MRI images may include analyzing the MRI images to separate fat and water in the aorta and defining vessel boundaries for segmentation.

The method for quantifying blood flow volume based on a blood flow model may further include: comparing a blood flow velocity vector of the generated standard blood flow model with a blood flow velocity vector of a 4D flow MRI image of a subject; and displaying, based on a result of the comparison, an abnormal blood flow region of the subject on the 4D flow MRI image of the subject.

The step of comparing the blood flow velocity vector of the generated standard blood flow model with the blood flow velocity vector of the 4D flow MRI image of the subject may include comparing an angle between the blood flow velocity vector of the standard blood flow model and the blood flow velocity vector of the 4D flow MRI image of the subject to determine whether the angle exceeds a predetermined angular threshold.

The step of displaying an abnormal blood flow region of the subject on the 4D flow MRI image of the subject based on a result of the comparison may include visually displaying an analysis result obtained by interpolating the 4D flow MRI image of the subject into a plurality of time steps at predetermined intervals.

According to the present disclosure, a blood flow model for 4D flow MRI, generated based on data collected in advance from healthy individuals, is used to quantify a subject's the blood flow. As a result, vascular diseases in the subject may be diagnosed, and treatment plans may be established using 4D flow MRI, without the complexity of data post-processing and interpretation.

Additional advantages, features, and aspects of the present disclosure will be apparent to those of ordinary skill in the art from the following detailed description and accompanying drawings, as well as from the appended claims.

### BRIEF DESCRIPTION OF THE DRAWINGS

FIG. 1 is a a block diagram illustrating a system for quantifying blood flow volume based on a blood flow model according to an example of the present disclosure.

FIG. 2 is a diagram illustrating an example of a method for quantifying blood flow volume based on a blood flow model according to an example of the present disclosure.

FIG. 3 is a diagram illustrating an example of vector classification during blood flow volume quantification based on a blood flow model according to an example of the present disclosure.

FIG. 4 is a diagram illustrating another example of blood flow volume quantification based on a blood flow model according to another example of the present disclosure.

FIG. 5 is a flowchart illustrating a method for quantifying blood flow volume based on a blood flow model according to an example of the present disclosure.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known after an understanding of the disclosure of this application may be omitted for increased clarity and conciseness, noting that omissions of features and their descriptions are also not intended to be admissions of their general knowledge.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application. The use of the term “may” herein with respect to an example or embodiment, e.g., as to what an example or embodiment may include or implement, means that at least one example or embodiment exists where such a feature is included or implemented, while all examples are not limited thereto.

Throughout the specification, when an element, such as a layer, region, or substrate, is described as being “on,” “connected to,” or “coupled to” another element, it may be directly “on,” “connected to,” or “coupled to” the other element, or there may be one or more other elements intervening therebetween. In contrast, when an element is described as being “directly on,” “directly connected to,” or “directly coupled to” another element, there can be no other elements intervening therebetween.

As used herein, the term “and/or” includes any one and any combination of any two or more of the associated listed items.

Although terms such as “first,” “second,” and “third” may be used herein to describe various members, components, regions, layers, or sections, these members, components, regions, layers, or sections are not to be limited by these terms. Rather, these terms are only used to distinguish one member, component, region, layer, or section from another member, component, region, layer, or section. Thus, a first member, component, region, layer, or section referred to in examples described herein may also be referred to as a second member, component, region, layer, or section without departing from the teachings of the examples.

Spatially relative terms such as “above,” “upper,” “below,” and “lower” may be used herein for ease of description to describe one element's relationship to another element as shown in the figures. Such spatially relative terms are intended to encompass different orientations of the device in use or operation in addition to the orientation depicted in the figures. For example, if the device in the figures is turned over, an element described as being “above” or “upper” relative to another element will then be “below” or “lower” relative to the other element. Thus, the term “above” encompasses both the above and below orientations depending on the spatial orientation of the device. The device may also be oriented in other ways (for example, rotated 90 degrees or at other orientations), and the spatially relative terms used herein are to be interpreted accordingly.

The terminology used herein is for describing various examples only, and is not to be used to limit the disclosure. The articles “a,” “an,” and “the” are intended to include the plural forms as well, unless the context clearly indicates otherwise. The terms “comprises,” “includes,” and “has” specify the presence of stated features, numbers, operations, members, elements, and/or combinations thereof, but do not preclude the presence or addition of one or more other features, numbers, operations, members, elements, and/or combinations thereof.

Due to manufacturing techniques and/or tolerances, variations of the shapes shown in the drawings may occur. Thus, the examples described herein are not limited to the specific shapes shown in the drawings, but include changes in shape that occur during manufacturing.

The features of the examples described herein may be combined in various ways as will be apparent after an understanding of the disclosure of this application. Further, although the examples described herein have a variety of configurations, other configurations are possible as will be apparent after an understanding of the disclosure of this application.

A detailed description is given below, with reference to attached drawings.

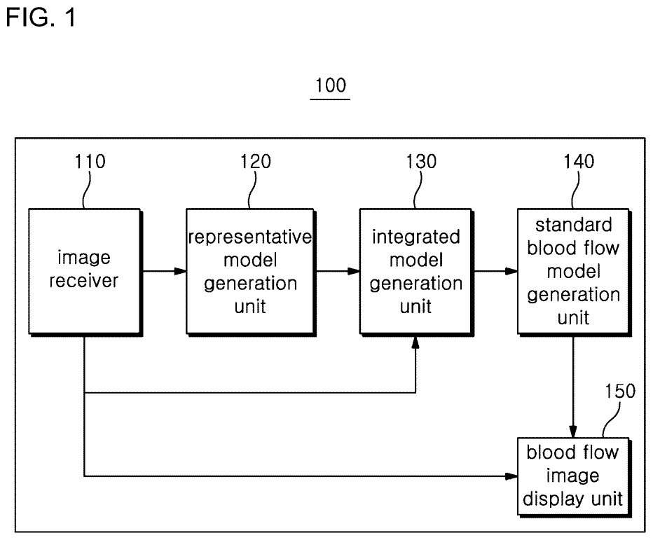

FIG. 1 is a block diagram illustrating a system for quantifying blood flow volume based on a blood flow model according to an example of the present disclosure.

Referring to FIG. 1, a blood flow volume quantification system 100 based on a blood flow model according to an example of the present disclosure (hereinafter referred to as the “blood flow volume quantification system”) includes an image receiver 110, a representative model generation unit 120, an integrated model generation unit 130, and a standard blood flow model generation unit 140. The blood flow volume quantification system may further include a blood flow image display unit 150.

The image receiver 110 of the blood flow volume quantification system 100 receives 4D flow MRI images and MRI images of a plurality of non-diseased individuals. In addition, the image receiver 110 of the blood flow volume quantification system 100 may receive 4D flow MRI images and/or MRI images of a subject who is either diagnosed with or suspected of having a disease, as will be described in more detail below.

4D flow MRI is a technique that adds time as a fourth dimension to three-dimensional spatial data by capturing spatial changes over time, thereby enabling visualization of the dynamic movement of blood flow and temporal tracking of blood flow velocity, direction, and volume. This technique measures time-varying blood flow in three-dimensional space by utilizing the inherent sensitivity of MRI to blood flow. Because it is based on three-dimensional measurements, it enables comprehensive cardiovascular assessment and retrospective analysis of blood flow velocity and volume in any blood vessel and from any angle. Unlike conventional 2D phase-contrast MRI (PC-MRI), which required precise selection of the imaging plane, 4D flow MRI allows for a single measurement of the entire vascular system, with subsequent analysis of the desired regions.

Dixon imaging is an MRI technique that clearly visualizes the distribution of fat and water in the body by separating the two. It separates the signals generated by fat and water using their chemical shift differences. Accordingly, Dixon imaging is highly effective in distinguishing fatty tissue from non-fatty tissue, thereby assisting in disease diagnosis and enabling clear visualization of anatomical structures.

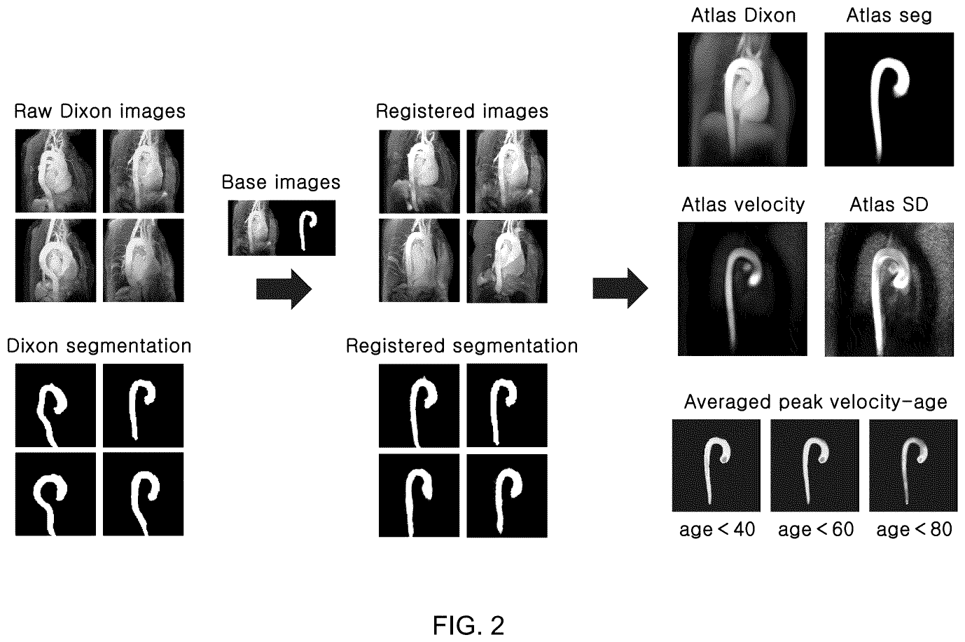

MRI imaging is particularly useful in the diagnosis of musculoskeletal disorders, liver diseases, and various types of cancer. In addition, MRI imaging can be effectively used to clearly identify specific tissues, such as perivascular fat. In the examples described below, MRI images targeting the aorta, as illustrated in FIG. 2, are used as examples. However, the present disclosure is not limited thereto and may also be applied to MRI images of various body regions of interest, including, but not limited to, other blood vessels such as the carotid artery and cerebral vessels.

According to an example of the present disclosure, the representative model generation unit 120 of the blood flow volume quantification system 100 performs vessel segmentation to distinguish blood vessels from surrounding tissues by separating fat and water signals in the received plurality of MRI images. That is, the MRI images are analyzed to separate fat and water in the aorta and to establish vessel boundaries for segmentation. The representative model generation unit 120 designates a representative aortic model from among the MRI images including a segmented aorta, based on predetermined criteria. The predetermined criterion for designating the representative model may include, but is not limited to, selecting the model with the median aortic diameter from the cohort of non-diseased individuals, selecting a model with geometry that is closest to a standard anatomical atlas, or selecting a model that minimizes the average spatial transformation required to register all other non-diseased individuals' images.

In other words, the representative model generation unit 120 selects an MRI image, which is determined to be representative, from among MRI images including an aorta of a subject identified as a healthy individual (hereinafter referred to as a “non-diseased individual”) according to predetermined criteria (corresponding to the base image in FIG. 2). The criteria for identifying a non-diseased individual and for determining the representative image in the blood flow volume quantification system 100 may be predetermined based on the intended implementation.

The integrated model generation unit 130 of the blood flow volume quantification system 100 generates an integrated model by registering the designated representative model with MRI images of the same anatomical region from other non-diseased individuals. That is, the integrated model generation unit 130 may generate an integrated aortic model by performing registration between the selected representative model and the aortic MRI images of other healthy donors. Through this process, as illustrated in FIG. 2, all MRI images of the non-diseased individuals are spatially aligned according to a consistent criterion, thereby facilitating comparison and analysis of the data.

In the registration process of the examples of the present disclosure, spatial registration techniques may be employed, and the image data of each non-diseased individual may be transformed to match the spatial orientation of the representative model. During this process, registration algorithms, such as affine transformation or non-linear deformation, may be applied so that the aortic image of each non-diseased individual is adjusted to closely resemble the representative model.

The standard blood flow model generation unit 140 of the blood flow volume quantification system 100 registers the received 4D flow MRI images of the plurality of non-diseased individuals to the generated integrated model to generate a standard blood flow model (also referred to as an “ATLAS”).

That is, in the generated standard blood flow model, each non-diseased individual's 4D flow MRI image may also be registered to the representative model along with the spatially aligned MRI images, thereby enabling all image data to be analyzed based on a consistent criterion. In addition, as illustrated in FIG. 2, during the generation of the standard blood flow model, an ATLAS Dixon and a segmentation image representing the final shape and segmented form of the aorta used in the model generation process may be generated; an ATLAS velocity image representing the aortic blood flow velocity calculated based on the standard blood flow model may be generated; and an ATLAS standard deviation (SD) image visualizing the variation range of blood flow velocity based on the standard deviation of the velocity data may also be generated.

According to an example of the present disclosure, the blood flow image display unit 150 of the blood flow volume quantification system 100 may compare the blood flow velocity vector of the generated standard blood flow model with the blood flow velocity vector of a subject's 4D flow MRI image (where the subject refers to a patient or a suspected patient for diagnosis and treatment), and may display abnormal blood flow regions of the subject on the subject's 4D flow MRI image based on the comparison result.

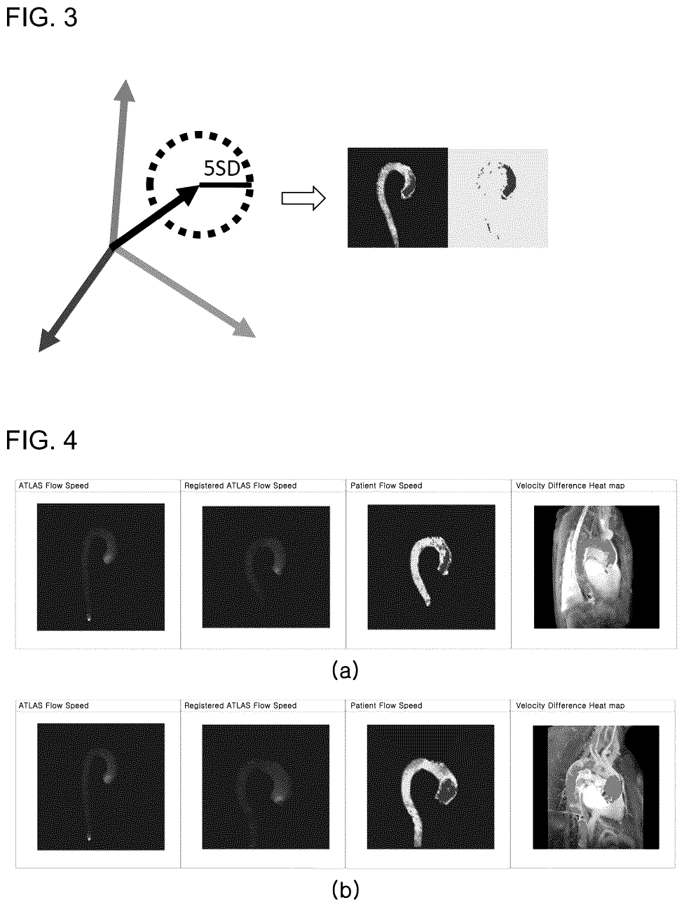

Specifically, the blood flow image display unit 150 according to an example of the present disclosure may classify the blood flow velocity vectors of a subject's 4D flow MRI image according to angular deviation, by comparing them with the mean (ATLAS mean) velocity vector of the generated standard blood flow model, as illustrated in FIG. 3. That is, by comparing the subject's 4D flow MRI image with the standard blood flow model, regions in which the vectors deviate from the mean by more than +5 standard deviations (SD) may be regarded as abnormal regions.

In addition, the blood flow image display unit 150 according to an example of the present disclosure may visualize abnormal blood flow characteristics based on the angular difference between the blood flow vectors of the standard blood flow model and those of the subject's data. That is, the mean vector of the standard blood flow model represents the general direction of blood flow, and the blood flow velocity vectors of the subject's 4D flow MRI image may be expressed in terms of angular deviation from the mean vector of the standard blood flow model.

In the blood flow volume quantification system 100 according to an example of the present disclosure, the blood flow velocity vectors of a subject's 4D flow MRI image may be classified into the following three angular ranges, as illustrated in FIG. 3.

0°<angle <60°: This range indicates a direction that is nearly aligned with, or slightly deviated from, the mean vector direction of the standard blood flow model.

60°<angle <120°: This range indicates a direction that is considerably deviated from the mean vector direction of the standard blood flow model.

120°<angle <180°: This range indicates a direction that is nearly opposite to the mean vector direction of the standard blood flow model.

The blood flow image display unit 150 according to an example of the present disclosure may display the vectors falling within the above three angular ranges in different colors to facilitate visual distinction, thereby allowing the classification results to be intuitively understood. Such visual representation in the blood flow image display unit 150 is useful for identifying abnormal blood flow in a patient by analyzing the direction and velocity of blood flow, and for assisting in diagnosis and treatment planning by comparing the results with the data of the standard blood flow model.

In addition, the blood flow image display unit 150 according to an example of the present disclosure may visually display the analysis results by interpolating a subject's 4D flow MRI image into a plurality of time steps at predetermined intervals. That is, the blood flow volume quantification system 100 according to an example of the present disclosure may perform interpolation by calculating and inserting new image data points between temporally spaced MRI scan images, thereby enabling estimation of the blood flow state at intermediate time points that were not actually measured. For example, the blood flow volume quantification system 100 may divide one cardiac cycle into 40 time steps, each representing a specific point in the cycle, thereby providing detailed information that was not included in the original image data.

The blood flow image display unit 150 according to an example of the present disclosure may convert the data of each generated time step into a visual image representing dynamic changes in blood flow and display the image. These images may visually represent blood flow characteristics, such as velocity, direction, and abnormalities, using color, brightness, vector arrows, or the like, thereby allowing intuitive understanding. The visual data presented in this manner may assist medical professionals in performing detailed analysis of the patient's blood flow patterns, identifying regions of abnormal flow, and evaluating how such abnormalities may affect disease progression.

For example, as illustrated in FIG. 3, the blood flow image display unit 150 according to an example of the present disclosure may visualize the average blood flow pattern of the standard blood flow model by representing the direction and velocity of blood flow using colors and streamlines. In addition, by comparing the subject's blood flow pattern with the mean of the standard blood flow model, regions in which the blood flow velocity vectors deviate by more than 5 standard deviations (5SD) from the mean vector of the standard blood flow model may be indicated as abnormal flow regions.

In addition, the integrated model generation unit 130 of the blood flow volume quantification system 100 according to an example of the present disclosure may generate integrated models for different age groups by calculating the average blood flow velocity and standard deviation based on the received 4D flow MRI images and MRI images of non-diseased individuals, as illustrated in FIG. 2. This allows for analysis of age-specific blood flow patterns.

Specifically, 4D flow MRI and MRI images of non-diseased individuals across various age groups may be collected. Blood vessels, such as the aorta, may be segmented from the MRI images to generate individual models. A representative model (also referred to as a ‘base image’) may be designated from among the non-diseased individuals, and other non-diseased individuals' aortic models may be registered to this representative model to generate an integrated model. Then, based on the MRI-aligned data, the corresponding 4D flow MRI data may also be registered to construct an age-specific standard blood flow model. That is, by calculating the average blood flow velocity and standard deviation of the collected 4D flow MRI data for each age group, a standard blood flow model for each age group may be constructed. The integrated models generated through this process may represent healthy blood flow patterns for each respective age group.

The following describes a method for quantifying blood flow volume based on blood flow modeling according to an example of the present disclosure, based on the foregoing description.

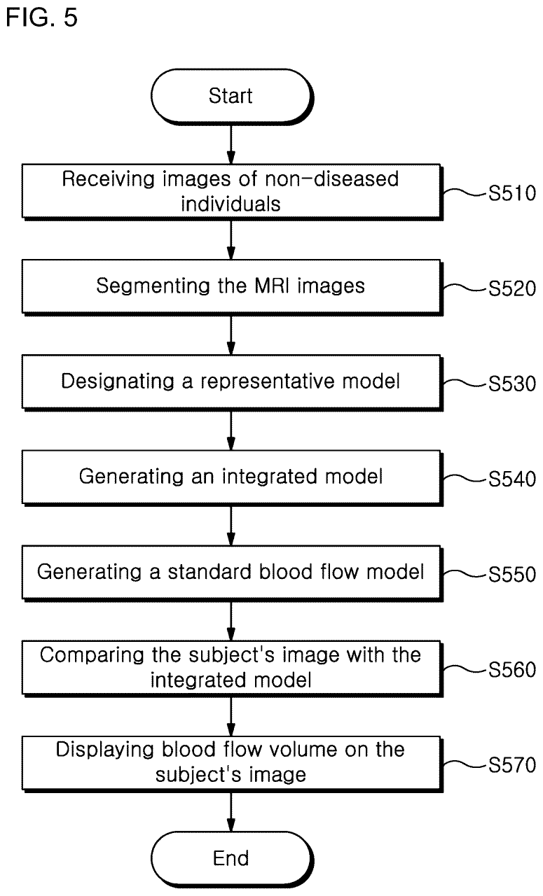

FIG. 5 illustrates a method for quantifying blood flow volume based on blood flow modeling according to an example of the present disclosure, which is an example implementation of the method using the blood flow volume quantification system based on blood flow modeling shown in FIG. 1.

Referring to FIG. 5, in the method for quantifying blood flow volume based on blood flow modeling according to an example of the present disclosure, an image receiver 110 receives 4D flow MRI images and MRI images of a plurality of non-diseased individuals (S510).

A representative model generation unit 120 performs vessel segmentation based on the received MRI images (S520), and designates a representative model based on a predetermined criterion from among the MRI images in which vessel segmentation has been performed (S530).

In this case, when the representative model generation unit 120 performs vessel segmentation based on the MRI images, it may analyze the MRI images to separate fat and water in the aorta and define the vessel boundaries for segmentation.

A representative model and MRI image representative models of other non-diseased individuals are registered by the integrated model generation unit 130 to generate an integrated model (S540), and the 4D flow MRI images are registered to the generated integrated model to generate a standard blood flow model, as illustrated in (a) of FIG. 4 (S550).

In addition, the blood flow velocity vector of the standard blood flow model generated by the integrated model generation unit 130 is compared with the blood flow velocity vector of the subject's 4D flow MRI image (S560), and, based on the comparison result, an abnormal blood flow region of the subject is displayed on the subject's 4D flow MRI image, as illustrated in (b) of FIG. 4 (S570).

In this case, when comparing the velocity vector of the standard blood flow model with the blood flow velocity vector of the subject's 4D flow MRI image, it is possible to determine whether the angular difference between the two vectors exceeds a predetermined angular threshold.

In addition, when displaying the abnormal blood flow region of the subject on the subject's 4D flow MRI image, the analysis results may be visually presented by interpolating the subject's 4D flow MRI image into a plurality of time steps at predetermined intervals.

Specifically, in order to accurately capture the complex patterns of blood flow and analyze the movement of blood flow, each time point in the 4D flow MRI data may be divided into multiple time steps, and an interpolation process may be performed to increase temporal resolution and subdivide a flow cycle, thereby enabling precise visualization of blood flow at each time step to represent natural blood flow motion.

As described above, the present disclosure enables the quantification of blood flow volume in a subject diagnosed with or suspected of having a disease, without the complexity of data post-processing or interpretation, by using a standard blood flow model—i.e., a blood flow model for 4D flow MRI generated based on data collected in advance from a plurality of non-diseased individuals—thereby allowing the diagnosis and treatment planning of vascular-related diseases using 4D flow MRI.

Additionally, the present disclosure is not limited to 4D flow MRI images and MRI images including the aorta as described above, but may also be applied to 4D flow MRI images and MRI images of various body regions depending on the subject of disease diagnosis and treatment.

The above-described embodiments may be implemented using various types of computing means that include at least one processor, memory, and storage medium. The computing means may also include a network interface connected to a wired or wireless network. The processor may be a central processing unit (CPU) or a semiconductor device configured to execute processing instructions stored in the memory and/or the storage unit. The memory and the storage unit may include volatile or non-volatile storage media. For example, the memory may include a ROM and a RAM. Accordingly, embodiments of the present disclosure may be implemented as a method executed by a computer or as a non-transitory computer-readable medium having computer-executable instructions stored thereon. In one embodiment of the present disclosure, when executed by the processor, the computer-readable instructions may perform a method according to at least one aspect of the present disclosure.

While the present disclosure has been described with reference to the illustrated embodiments, these embodiments are merely exemplary and not intended to limit the scope of the present disclosure. It will be apparent to those of ordinary skill in the art that various modifications, changes, and equivalent embodiments may be made without departing from the spirit and scope of the present disclosure. For example, the representative model generation unit 120 and the integrated model generation unit 130 may be implemented as a single integrated module, or alternatively, they may be implemented as two or more separate devices. Therefore, the true scope of the technical protection of the present disclosure should be defined by the spirit of the appended claims.

What is claimed is:

1. A blood flow volume quantification system based on a blood flow model, comprising:
an image receiver configured to receive 4D flow magnetic resonance imaging (MRI) images and MRI images of a plurality of non-diseased individuals;
a representative model generation unit configured to perform vessel segmentation based on the received MRI images and to designate a representative model from among the MRI images in which vessel segmentation has been performed, based on a predetermined criterion;
an integrated model generation unit configured to register MRI images of other non-diseased individuals to the representative model to generate an integrated model; and
a standard blood flow model generation unit configured to register the 4D flow MRI images to the integrated model to generate a standard blood flow model,
wherein the 4D flow MRI images and the MRI images are images of a body region captured for diagnostic purposes.

2. The blood flow volume quantification system based on a blood flow model of claim 1,
wherein the representative model generation unit is configured to analyze the MRI images to separate fat and water in the aorta, and segments the vessels by defining vessel boundaries based on the separation.

3. The blood flow volume quantification system based on a blood flow model of claim 1, further comprising:
a blood flow image display unit configured to compare a velocity vector of the generated standard blood flow model with a blood flow velocity vector of a 4D flow MRI image of a subject, and to display, based on a result of the comparison, an abnormal blood flow region of the subject on the 4D flow MRI image of the subject.

4. The blood flow volume quantification system based on a blood flow model of claim 3,
wherein the blood flow image display unit is configured to compare an angle between the velocity vector of the standard blood flow model and the blood flow velocity vector of the 4D flow MRI image of the subject, and to determine whether the angle exceeds a predetermined angular threshold.

5. The blood flow volume quantification system based on a blood flow model of claim 3,
wherein the blood flow image display unit is configured to visually display an analysis result obtained by interpolating the 4D flow MRI image of the subject into a plurality of time steps at predetermined intervals.

6. A method for quantifying blood flow volume based on a blood flow model, the method comprising:
receiving 4D flow magnetic resonance imaging (MRI) images and MRI images of a plurality of non-diseased individuals;
performing vessel segmentation based on the received MRI images;
designating a representative model based on a predetermined criterion from among the MRI images in which vessel segmentation has been performed;
registering MRI images of other non-diseased individuals to the representative model to generate an integrated model; and
registering the 4D flow MRI images to the generated integrated model to generate a standard blood flow model.

7. The method for quantifying blood flow volume based on a blood flow model of claim 6,
wherein the vessel segmentation based on the MRI images comprises analyzing the MRI images to separate fat and water in the aorta and defining vessel boundaries for segmentation.

8. The method for quantifying blood flow volume based on a blood flow model of claim 6, further comprising:
comparing a blood flow velocity vector of the generated standard blood flow model with a blood flow velocity vector of a 4D flow MRI image of a subject; and
displaying, based on a result of the comparison, an abnormal blood flow region of the subject on the 4D flow MRI image of the subject.

9. The method for quantifying blood flow volume based on a blood flow model of claim 8,
wherein the comparison of the blood flow velocity vector of the generated standard blood flow model with the blood flow velocity vector of the 4D flow MRI image of the subject comprises comparing an angle between the blood flow velocity vector of the standard blood flow model and the blood flow velocity vector of the 4D flow MRI image of the subject to determine whether the angle exceeds a predetermined angular threshold.

10. The method for quantifying blood flow volume based on a blood flow model of claim 8,
wherein the displaying of an abnormal blood flow region of the subject on the 4D flow MRI image of the subject based on a result of the comparison comprises visually displaying an analysis result obtained by interpolating the 4D flow MRI image of the subject into a plurality of time steps at predetermined intervals.
